(19) United States
(12) Patent Application Publication
(10) Pub. No.: US 2009/0169502 A1
(43) Pub. Date: Jul. 2, 2009

(54) COMPOSITIONS AND METHODS FOR TREATING KERATINOUS SUBSTRATES USING POLYAMIDES

(75) Inventor: Murat Quadir, Midlowthian, VA (US)

Correspondence Address: Connolly Bove Lodge & Hutz LLP;Suite 1100, 1875 Eye Street, NW, Washington, DC, 20006, US

(73) Assignee: L'Oreal, Paris (FR)

(21) Appl. No.: 12/347,666

(22) Filed: Dec. 31, 2008

Related U.S. Application Data
(60) Provisional application No. 61/018,583, filed on Jan. 2, 2008.

(51) Int. Cl. A61K 8/88; A61Q 5/12

(52) U.S. Cl. 424/70.9; 424/70.17

(57) ABSTRACT

The disclosure relates to compositions and methods of using the compositions to treat keratinous substrates. The compositions provide a water resistant and non-transferable protective barrier on the substrate. For example, the compositions provide hair with improved shine, condition, manageability and color. The compositions contain at least one polyamide, at least one monoacid, at least one water-insoluble ingredient, solvent and optionally at least one auxiliary ingredient. The methods for treating keratinous substrates involve contacting the keratinous substrates with the compositions of the disclosure.

### TECHNICAL FIELD

The disclosure relates to compositions and methods for treating keratinous substrates. The compositions and methods provide a water resistant and non-transferable protective barrier on keratinous substrates imparting the substrates with improved properties.

### BACKGROUND OF THE DISCLOSURE

When keratinous substrates are exposed to environmental conditions, the substrates can lose many of their desirable properties. For example, hair can lose its shine, it can become unmanageable, it can lose its color and it can become brittle. One method of maintaining these desirable properties is to provide a protective barrier on keratinous substrates like hair. For example, under low humidity conditions hair can dry out and dried-out hair tends to be less shiny and more brittle. A protective moisture barrier on the hair will help to keep moisture in the hair allowing hair to keep its shine. Conversely, under high humidity conditions hair tends to absorb water causing hair to lose its shape and become unmanageable and unattractive. A protective moisture barrier on the hair will help keep moisture out of the hair under high humidity conditions leading to improved manageability. Such a protective barrier can also inhibit color fading in both dyed and naturally colored hair. This protective barrier can be applied to other keratinous substrates such as skin, lips., nails and eyelashes. The protective barrier is also useful in cosmetic applications such as makeup, skin care and sun care products. Such a protective barrier should be water-resistant so that the barrier is not easily removed. In addition, the protective barrier should not be easily transferred from the substrate over time by normal everyday activity. Accordingly, a product that provides a protective barrier to the substrate that also is water resistant and non-transferable would be of benefit to the area of cosmetic products.

### BRIEF SUMMARY OF THE DISCLOSURE

The disclosure relates to compositions for treating keratinous substrates and methods of using the compositions to treat hair. The disclosed compositions provide a water resistant and non-transferable protective barrier on the keratinous substrates imparting desirable properties to the substrate. The methods involve applying the compositions to the substrates. The compositions contain at least one polyamide, at least one monoacid, at least one water-insoluble ingredient, solvent and optionally at least one auxiliary ingredient. The methods for imparting desirable properties to the keratinous involve contacting the substrate with the compositions of the disclosure. Methods for improving the properties of keratinous substrates are also disclosed. Specifically, methods for improving the shine, condition and manageability of hair are disclosed. In addition, methods of inhibiting color fading in both dyed and naturally colored hair are also disclosed.

### DETAILED DESCRIPTION OF THE DISCLOSURE

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Other than in the operating examples, or where otherwise indicated, all numbers expressing quantities of ingredients and/or reaction conditions are to be understood as being modified in all instances by the term “about”.

The term “water-insoluble” means those compounds which are either completely or partially insoluble in water.

“At least one” as used herein means one or more and thus includes individual components as well as mixtures/combinations.

“Conditioning” as used herein means imparting to at least one keratinous fiber at least one property chosen from combability, manageability, moisture-retentivity, luster, shine, and softness. The state of conditioning is evaluated by measuring, and comparing, the ease of combability of the treated hair and of the untreated hair in terms of combing work (gm-in).

Amino groups include primary amino groups, secondary amino groups and tertiary amino groups and further includes amino groups which are terminal, pendant and intercalated in a skeleton of the polyamine compound. The polyamine compounds disclosed include at least two amino groups.

In an embodiment of the disclosure the composition for treating keratinous substrates comprises:
(a) at least one polyamide,
(b) at least one monoacid,
(c) at least one water-insoluble ingredient and
(d) solvent, wherein the at least one polyamide is at least one selected from the group consisting of

1) a polyamide formed by an amidation reaction between a polyamine and a lactone wherein the molar ratio of the amine groups of the polyamine to the carboxylate groups of the lactone is from about 2 to 1 to about 60 to 1,

2) a poly di-C₁₋₆-alkyl amino-C₁₋₆-alkyl (meth)acrylamide-co-poly (poly-C₁₋₆-alkylglycol (meth)acrylate) polymer and

3) a poly (2-substituted-2-oxazoline) polymer wherein from about 3 to about 97 mol % of the amide groups in the poly (2-substituted-2-oxazoline) polymer are hydrolyzed to give amine groups.

Generally, the polyamide (a) contains amine group as well as amide groups. The amines are primary (—NH₂) or secondary (—NH) amines that can complex with the at least one monoacid (b) of the disclosed composition. The ratio of amide groups to amine groups can vary depending upon the polyamide structure.

When the polyamide 1) is utilized, the polyamine may, for example, be chosen from a polyethyleneimine which may be linear or branched, a polyvinylamine, an aminated polysaccharide, an amine substituted polyalkylene glycol, an amine substituted polyacrylate crosspolymer, an amine substituted polyacrylate, an amine substituted polymethacrylate, an aminosilicone, a protein, an amine substituted polyester, a polyamino acid, an amodimethicone, a polyalkylamine, diethylene triamine, triethylenetetramine, spermidine, spermine and mixtures thereof.

Non-limiting examples of polyethyleneimine include Lupasol™ products commercially available from BASF. Suitable examples of Lupasol™ polyethyleneimines include Lupasol™ PS, Lupasol™ PL, Lupasol™ PR8515, Lupasol™ G20, Lupasol™ G35 as well as Lupasol™ SCE® Polyethyleneimine Reaction Products (such as Lupasol™ SC-61B, Lupasol™ SC-62J®, and Lupasol™ SC-86X®). Other non-limiting examples of polyethyleneimines which may be used in the composition according to the present invention are the Epomin™ products commercially available from Aceto. Suitable examples of Epomin™ polyethyleneimines include Epomin™ SP-006, Epomin™ SP-012, Epomin™ SP-018, and Epomin™ P-1000. These examples include substituted polyethyleneimines.

Non-limiting examples of polyvinylamines include Lupamines® 9095, 9030, 9010, 5095 and 1595 from BASF.

An example of an amine substituted polyalkylene glycol includes PEG-15 cocoloryamine from Cognis.

An example of an aminosilicone includes DC 2-8566, an amino functional polydimethylsiloxane fluid from Dow Corning®.

In another embodiment, the polyamine compound is chosen from proteins and protein derivatives. Non-limiting examples of suitable proteins and protein derivatives for use in the present invention include those listed at pages 1701 to 1703 of the C.T.F.A. International Cosmetic Ingredient Dictionary and Handbook, 8^{th} edition, vol. 2, (2000) (incorporated herein by reference). In one embodiment, the at least one polyamine compound is chosen from wheat protein, soy protein, oat protein, collagen, and keratin protein.

In another embodiment, the polyamine compound is chosen from compounds comprising lysine, compounds comprising arginine, compounds comprising histidine, and compounds comprising hydroxylysine. Not limiting examples include chitosan and polylysine.

An example of an amine substituted polyacrylate crosspolymer includes Carbopol® Aqua CC polymer from Noveon, Inc.

When the polyamide 1) is utilized, the lactone used is not limited and may be selected from, from the group consisting of butyrolactone, butyrolactonethiol, gamma-caprolactone, delta-decalactone, gamma-decalatone, ethyl butyl valerolactone, galactonolactone, glucarolactone, glucoheptonolactone, gluconolactone, glucuronolactone, hexadecanolactone, meadowfoam delta-lactone(Meadowlactone™ from Fancor Ltd, Chicago, Ill.), mevalonolactone, gamma-nonalactone, pantolactone, pentadecalactone, ribonolactone, gamma-undecalactone and mixtures thereof.

The polyamide 1) is prepared by an amidation reaction between the polyamine and lactone react at temperatures between 30° C. and 100° C. at ambient pressure to form the polyamide. The amount of lactone utilized can be varied to give the desired properties and the desired amount of residual amine groups. The molar ratio of amine groups from the polyamide to the carboxylate groups from the lactone (which from the amide groups) is from about 2 to 1 to about 60 to 1. Typically, the molar ratio is from about 4 to 1 to about 40 to 1 and more typically from about 8 to 1 to about 20 to 1.

The polyamide 2) may be selected from poly (di-C₁₋₆-alkylamino-C₁₋₆-alkyl (meth)acrylamide-co-poly (poly-C₁₋₆-alkylglycol (meth)acrylate) polymers. This class of polymers has been described in WO/2007/003284, WO/2006/013268, WO/2006/013271 and WO/2006/013269, the contents of each are herein incorporated by reference. Typically, the C₁₋₆ moiety is methyl, ethyl, propyl, isopropyl, butyl and isobutyl. The polyalkylglycol moiety is typically 1 to 20 repeating units and more typically 2 to 10 repeating units. A typical polyamide polymer is poly (dimethylaminopropylmethacrylamide)co-poly-(polyethylene glycol (meth)acrylate) polymer.

The polyamide 3) is selected from poly (2-substituted-2-oxazoline) polymer where from about 3 to about 97 mol % of the amide groups in the polymer are hydrolyzed to give amine groups. The substituent on the polymer is not limited. The polymer may be substituted at the two-position with a C₁-C₃₀ alkyl group that may be a straight chain, branched or cyclic alkyl. The polymer may also be substituted with a C₆-C₃₀ alkylaryl, a C₆-C₃₀ arylalkyl, a C₆-C₃₀ alkyl alkylheteroaryl, a C₆-C₃₀ heteroarylalkyl or a phenyl group optionally substituted with one or more C₁-C₆ alkyl groups. Typically, the unhydrolyzed polyamide in polyamide 3) is poly (2-ethyl-2-oxazoline) polymer or poly (2-phenyl-2-oxazoline) polymer.

In other embodiments, the substituent group may be hydrophobic, hydrophilic, anionic, cationic or amphoteric. Non-limiting groups include methyl, nonyl, a C₁-C₃₀ alkanol, a C₁-C₃₀ alkylcarboxylic acid and its alkali metal salts, a C₁-C₃₀ alkylpolyol, a C₁-C₃₀ alkylquaternary amine, a C₁-C₃₀ alkylsulfonate, a C₁-C₃₀ alkylsulfate, a C₁-C₃₀ alkylphosphonate, a C₁-C₃₀ alkyl phosphate, a polysiloxane group or an amino acid group.

The polyamide 3) polymer may be prepared as described in Journal of Polymer Science: Part A=Polymer Chemistry, Vol. 45, 416-422 (2007), U.S. Pat. No.4,8837,005 and EP 0 262 641, the contents of which are hereby incorporated by reference. The polymer typically has a narrow molecular weight distribution and it can be a multi-block copolymer. The degree of hydrolysis of the amine groups of the polymer to give the amine groups can vary depending upon the desired properties and the desired number of amine groups. Typically, from about 3 to about 97 mol % of the amide groups in the polymer are hydrolyzed. More typically from about 10 mol % to about mol % 90 of the amide groups are hydrolyzed and even more typically, from about 15 mol % to about 85 mol % are hydrolyzed.

The unhydrolyzed polymer has a typical molecular weight in the range of about 5,000 to 500000 daltons. The poly-2-ethyloxazoline polymer may be obtained for example from Polymer Chemistry Innovations under the Trademark Aquazol®.

In the present disclosure, the polyamide a) is used in a positive amount up to about 30% by weight, more typically a positive amount up to about 10% by weight, and most typically a positive amount up to about 5% by weight, based on the weight of the composition as a whole. In some embodiments the at least one polyamine ranges from about 0.1% to about 30% by weight based on the weight of the composition. In other embodiments the at least one polyamine ranges from about 0.1 wt % to about 10 wt %, based on the weight of the composition as a whole and in further embodiments the range is from about 0.1 wt % to about 5 wt %.

The at least one monoacid (b) of the composition may, for example, be chosen from a fatty carboxylic acid, a fatty ether carboxylic acid, a fatty ether phosphoric acid, a fatty phosphoric acid and mixtures thereof.

Non-limiting examples of fatty carboxylic acids includes fatty acids having from about 6 to about 40 carbon atoms corresponding formula (I)

RCOOH (1)

wherein:

R is a hydrocarbon radical containing from about 6 to about 40 carbon atoms. In addition, R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, and even more typically a C₁₂₋₁₈ alkyl group or alkenyl group or a C₆₋₁₆ alkyl phenyl group.

Suitable fatty acids having from about 6 to about 40 carbon atoms include, but are not limited to the following representatives referred to by their INCI names (INCI: nomenclature for raw materials according to the International Cosmetic Ingredient Dictionary, 10^{th} Edition, published by the Cosmetic, Toiletry and Fragrance Association Inc. (CTFA), Washington D.C., USA): Arachidic Acid, Arachidonic Acid, Beeswax Acid, Capric Acid, Caproic Acid, Caprylic Acid, Coconut Acid, Isostearic Acid, Laurie Acid, Linoleic Acid, Linolenic Acid, Myristic Acid, Oleic Acid, Olive Acid, Palmitic Acid, Rapeseed Acid, Stearic Acid, Behenic Aid, Tallow Acid, Undecanoic Acid, Undecylenic Acid or Wheat Germ Acid and mixtures thereof:

Typical fatty acids having from about 6 to about 40 carbon atoms include Capric Acid, Caprylic Acid, Lauric Acid, Oleic Acid, Isostearic Acid, and Stearic Acid.

Non-limiting examples of fatty ether carboxylic acid includes compounds corresponding to formula (II):

RO[CH₂O]ᵤ[(CH₂)ₓCH(R′)(CH₂)_{y}(CH₂)_{z}O]ᵥ[CH₂CH₂O]_{w}CH₂COOH (II)

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z is ≧0;

Ether carboxylic acids corresponding to formula (II) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (II), R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkene group or a C₄₋₁₈ alkyl phenyl group, and even more typically a C₁₂₋₁₈ alkyl group or alkenyl group or a C₆₋₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable ether carboxylic acids or ether carboxylates include, but are not limited to, the following representatives referred to by their INCI names (INCI: nomenclature for raw materials according to the International Cosmetic Ingredient Dictionary, 7^{th} Edition, published by the Cosmetic, Toiletry and Fragrance Association Inc. (CTFA), Washington D.C., USA): Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉₋₁₁ Pareth-6 Carboxylic Acid, C₁₁₋₁₅ Pareth-7 Carboxylic Acid, C₁₂₋₁₃ Pareth-5 Carboxylic Acid, C₁₂₋₁₃ Pareth-8 Carboxylic Acid, C₁₂₋₁₃ Pareth-12 Carboxylic Acid, C₁₂₋₁₅ Pareth-7 Carboxylic Acid, C₁₂₋₁₅ Pareth-8 Carboxylic Acid, C₁₄₋₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof.

Typical Carboxylic Acids are Oleth-10 Carboxylic Acid, Laureth-5 Carboxylic Acid and Laureth-11 Carboxylic Acid.

Non-limiting examples of fatly phosphoric acids include compounds corresponding to Formula II:

R—O—P(O)(OH)₂ (III)

wherein:
R is a hydrocarbon radical containing from about 6 to about 40 carbon atoms. In addition, R is linear or branched, acyclic or cyclic, saturated or unsaturated, aliphatic or aromatic, substituted or unsubstituted. Typically, R is a linear or branched acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group and most typically a C₁₂₋₁₈ alkyl group or alkenyl group or a C₆₋₁₆ alkyl phenyl group.

Typical fatty phosphoric acids include capryl phosphate, caprylyl phosphate, lauryl phosphate, oleyl phosphate, isostearyl phosphate, stearyl phosphate and cetyl phosphate.

Non-limiting examples of fatty ether phosphoric acids compounds corresponding to formulas IV and V:

RO[CH₂O]ᵤ[(CH₂)ₓCH(R′)(CH₂)_{y}(CH₂)_{z}O]ᵥ[CH₂CH₂O]_{w}—PO—(OH)₂ Formula IV,

{RO[CH₂O]ᵤ[(CH₂)ₓCH(R′)(CH₂)_{y}(CH₂)_{z}O]ᵥ[CH₂CH₂O]_{w}}₂PO—(OH) Formula V

and combinations thereof;

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z being ≧0.

The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formulas IV and V, R is linear of branched, acyclic or cyclic, saturated or unsaturated, aliphatic or aromatic, substituted or unsubstituted, typically a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, even more typically a C₁₂₋₁₈ is alkyl group or alkenyl group or a C₆₋₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Typical fatty ether phosphoric acids include PPG-5-Ceteth-10 phosphate (CRODAFOS SG®), Oleth-3 phosphate (CRODAFOS N3 acid), Oleth-10 phosphate (CRODAFOS N10 acid), and a mixture of Ceteth-10 phosphate and Dicetyl phosphate (CRODAFOS CES) all sold by Croda.

The at least one monoacid (b) is present in the composition in a positive amount up to about 50% by weight, typically a positive amount up to about 30% by weight, and more typically a positive amount up to about 15% by weight, based on the weight of the composition as a whole. In other embodiments, the at least one monoacid (b) is present in the composition in a range of from about 2 to about 50% by weight and in a range from about 5 to about 15% by weight, based on the weight of the composition as a whole.

The at least one water-insoluble ingredient (c) may, for example, be chosen from an oil, a polymer, a fatty ester, a hydrocarbon, a silicone, a wax, a fatty acid (in addition to the fatty acid (a)), salts of fatty acids, a fatty alcohol and mixtures thereof.

Non-limiting examples of oils include plant oil such as olive oil, avocado oil, coconut oil, aloe vera oil, almond oil, castor oil, jojoba oil, peanut oil, sesame oil, hazelnut oil, sunflower oil, colza oil, grapeseed oil, linseed oil and palm oil.

Non-limiting examples of hydrocarbon oils include mineral oil, petrolatum, paraffins, iso-paraffins, aromatic hydrocarbons and C₁₀₋₄₀ hydrocarbons which may be aliphatic, aromatic, arylaliphatic or mixtures thereof and the aliphatic hydrocarbons may be straight chain, branched, cyclic or combinations thereof.

Non-limiting examples of silicones include phenyltrimethicone, dimethicone, cyclomethicone, dimethicone copolyol, aminosilicone, laurylmethicone copolyol, cetyl dimethicone, cetyl triethylammonium dimethicone copolyol phthalate, dimethicone copolyol lactate, silicone quaternium 13, stearalkonium dimethicone copolyol phthalate, stearaminopropyl dimethicone and polyorganosiloxanes such as polydimethylsiloxane.

Non-limiting examples of waxes include paraffin wax, beeswax, candelilla wax, carnauba wax, jasmine wax, jojoba wax and mimosa wax.

Non-limiting examples of fatty acids are the same as those described above for the at least one monoacid (b) described above. This includes carboxylate salts of the fatty acids listed above. The sodium, potassium, ammonium, calcium and magnesium carboxylates of the fatty acids listed above are typical examples of the carboxylate salts of the fatty acids.

Non-limiting example of fatty alcohols include compounds of formula (VI):

R—OH (VI)

where R is as described above for the at least one fatty acid.

Non-limiting fatty esters include esters formed from the fatty acid of formula (I) and C₁₋₁₀ alcohols and esters formed from the fatty alcohol of formula VI and C₁₋₁₀ carboxylic acids.

In addition, non-limiting specific examples of water-insoluble ingredients includes isopropyl palmitate, capric triglyceride, caprylic triglyceride, isodecane, polylsobutylene. Vitamin E, Vitamin E Acetate, Vitamin A, Vitamin A Palmitate, 2-oleamido-1,3-octadecanediol, octymethoxy cinnamate, octyl salicylate and mixtures thereof.

The at least one water-insoluble ingredient (c) is present in the composition in a positive amount up to about 50% by weight, typically a positive amount up to about 30% by weight, and more typically a positive amount up to about 15% by weight based on the weight of the composition as a whole. In other embodiments, the at least one water-insoluble ingredient (c) is present in the composition in an amount from about 0.1 to about 50% by weight and in an amount from about 0.5 to about 15% by weight based on the weight of the composition as a whole.

Solvent (d) in the composition is present in an amount from about 10% by weight to about 95% by weight, typically in an amount from about 50% by weight to about 85% by weight and more typically from about 60% by weight to 80% by weight, based on the weight of the composition as a whole. The solvent is typically water or an alcohol. Alcohols include ethanol, propanol and butanol. Typically, the alcohol is ethanol or isopropanol.

The composition may optionally contain at least one auxiliary ingredient (e). The auxiliary ingredient may include proteins, amino acids cationic conditioners, cationic polymers, nonionic surfactants, anionic surfactants, amphoteric surfactants, zwitterionic surfactants, viscosity modifiers, antibacterial agents, sunscreens, preservatives, PH adjusting agents, bleaching agents, perfumes, sequestering agents, anti-dandruff agents and mixtures thereof.

Non-limiting examples of proteins include collagen, deoxyribonuclease, iodized corn protein, milk protein, protease, serum protein, silk, sweet almond protein, wheat germ protein, wheat protein, alpha and beta helix of keratin proteins, hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Non-limiting examples of amino acids include amino acids derived from the hydrolysis of various proteins as well as the salts, esters, and acyl derivatives thereof. Non-limiting examples of such amino acid agents include amphoteric amino acids such as alkylamido alkylamines, i.e. stearyl acetyl glutamate, capryloyl silk amino acid, capryloyl collagen amino acids, capryloyl keratin amino acids, capryloyl pea amino acids, cocodimonium hydroxypropyl silk amino acids, corn gluten amino acids, cysteine, glutamic acid, glycine, hair keratin amino acids, amino acids such as asparatic acid, threonine, serine, glutamic acid, proline, glycine, alanine, cystine, valine, methionine, isoleucine, leucine, tyrosine, phenylalanine, cysteic acid, lysine, histidine, arginine, cysteine, tryptophan, citrulline, lysine, silk amino acids, wheat amino acids and mixtures thereof.

Non-limiting examples of cationic conditioners include quaternium 27, behenamidopropyl PG-dimonium chloride, hydroxyethyl tallowdimonium chloride, hexadimethrine chloride, stearalkonium chloride and cetrimonium chloride.

Non-limiting examples of cationic polymers include polyquaternium 4, polyquaternium 6, polyquaternium 7, polyquaternium 10, polyquaternium 11, polyquaternium 16, polyquaternium 22 and polyquaternium 32.

Non-limiting examples of nonionic surfactants includes alkoxylated derivatives of the following: fatty alcohols, alkyl phenols, fatty acids, fatly acid esters and fatty acid amides, wherein the alkyl chain is in the C₁₂₋₅₀ range, typically in the C₁₆₋₄₀ range, more typically in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), steareth-10 (a stearyl alcohol ethoxylate having an average degree of ethoxylation of 10), steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Commercially available nonionic surfactants are Brij® nonionic surfactants from Uniqema, Willmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, which are the condensation products of long chain alcohols, e.g. C₈-C₃ alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein sucrose ester surfactants such as sucrose cocoate and sucrose laurate.

Other nonionic surfactants suitable for use in the present invention are glycerly esters and polyglyceryl esters, including but not limited to, glyceryl monesters, typically glycerly monesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquiolcate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use as nonionic surfactants are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being ypical. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to 20).

Non-limiting examples of anionic surfactants include compounds in the classes known as alkyl sulfates, alkyl ether sulfates, alkyl sulfonates, alkyl ether sulfonates, sulfate esters of an alkylphenoxy polyoxyethylene ethanol, alpha-olefin sulfonates, beta alkyloxy alkene sulfonates, alkyl arylsulfonates, alkyl carbonates, alkyl ether carboxylates, fatty acids, succinamates, sulfosuccinates, sarcosinates, octoxynol or nonoxynol phosphates, taurates, fatty taurides, sulfated monoglycerides, fatty acid amino polyoxyethylene sulfates, isothienates and mixtures thereof. Specific examples of amionic surfactants include the ammonium, monoethanolamine, diethanolamine, triethanolamine, isopropylamine, sodium, potassium, lithium, or magnesium salts of lauryl sulfate, dodecylbenzene-sulfonate, lauryl sulfosuccinate, lauryl ether sulfate, lauryl ether carboxylate, lauryl sarcosinate, cocomethyl tauride, and sulfosuccinate half ester amide and mixtures thereof.

Non-limiting examples of amphoteric and zuitterionic surfactants include alkyl, alkyl dimethyl, alkylamido, alkyl amide, alkylamidopropyl, or alkyl dimethylammonium betaine; alky amidopropyl or alkyl sulfobetaine; alkyl, alkylampho, or alkylamphocarboxy glycinate; alkyl, or alkyl substituted imidazoline mono or dicarboxylate; sodium salts of alkyl mono-or dicarboxylates; alkyl beta amino acids; alkyl amidopropyl, or alkyl ether hydroxysultaine; alkyl amidopropyl dimethyl ammonia acetate; alkyl ampho mono-or diacetate; alkyl, or alkyl ampho, or alkyl imino dipropionate; alkyl amphopropionate; alkyl beta amino propionic acid; alkyl dipropionate; alkyl beta iminodipropionate; branched or n-alkyl dimethylamidopropionate; alkyl carboxylated propionate; alkyl, or methyl alkyl imidazoline; fluorinated alkyl amphoteric mixtures; and/or nonionic surfactants such as, but not limited to, alkyl, alkyl dimethyl, alkyl amidopropylamine, or bis 2-hydroxy ethyl alkyl amine oxides; alkanolamides; alkyl amides; polyoxyethylene glycol (PEG) of monoglycerides, of sorbitan esters, of branched or linear fatly alcohol ethers, of branched or linear fatly acid ethers, of thioethers; alkyl oxoalcohol PEG; PEG fatty esters; polyoxyethlyene glycol/polyoxpropylene glycol block copolymers; alkyl phenol PEG ethers; alkyl polyglucosides, or polysaccarides, polysiloxane polyethoxylene ether and mixtures thereof. Specific examples include cocamidopropyl betaine, lauramidopropyl betaine, coco/oleamidopropyl betaine, coco betaine, oleyl betaine, cocamidopropyl hydroxysultaine, tallowamidopropyl hydroxysultaine and dihydroxyethyl tallow glycinate and mixtures thereof.

Non-limiting examples of viscosity modifiers include water swellable/soluble cationic polymers from quaternized polysaccharides such as trimethyl ammonium substituted epoxide of hydroxyethyl cellulose, diallyl dimethyl ammonium salts of hydroxyethylcellulose, deacylated chitin or chitosan, dihydroxypropyl chitosan trimoniurn chloride, hydroxypropltrimethyl ammonium chloride guar, locust bean, or konjac mannan gum; quaternized synthetics such as acrylamide dimethyl diallyl ammonium chloride copolymers, acrylamide/dimethyl diallyl ammonium chloride/acrylic acid terpolymer, quaternized poly (vinyl pyrrolidone/dimethyl amino ethylmethacrylate), poly (vinylpyrrolidone/methacrylamidopropyl trimethylammonium chloride), polyvinyl pyrrolidone/methylvinylimidazolinium chloride or methyl sulfate copolymer, chloroethylether/dimethylaminopropylamine/adipate or azelate terpolymer, vinylpyrrolidone/methacrylamidopropyl trimethylammonium chloride, acrylonitrile/acrylic acid/dimethylpropanediammonium acrylates sulfate terpolymer. Anionic or nonionic polysaccharide polymers such as gum tragacanth, sodium or propylene glycol alginate, kappa-, iota-, or lambda-carrageenan, guar or hydroxyl propyl guar gum, karaya gum, gum Arabic, locust bean gum, konjac mannan gum, gellan, xanthan, succinoglycan or its acidic or enzymatic hydrolysates, sodium carboxymethyl cellulose, methycellulose, hydroxylethylcellulose, hydroxypropylmethylcellulose, and hydroxypropylecellulose; and/or hydrophobically modified anionic, cationic, or nonionic polymers such as, but not limited to, alkyl and/or substituted hydroxyethylcellulose, lauryl dimethyl ammonium substituted epoxide of hydroxyethylcellulose, propoxylated cellulosic, xanthan, succinoglycan, or polygalactomannoses, alkyl methacrylates/crosslinked acrylic acid copolymer and/or acrylonitrile/acrylates block copolymer.

Non-limiting examples of antibacterial agents include bacitracin, phenol, benzethonium chloride, erythromycin, neomycin, tetracycline, chlortetracycline and mixtures thereof.

Non-limiting examples of sunscreens include benzophenones, bornelone, butyl paba, cinnamidopropyl trimethyl ammonium chloride, disodium distryrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and mixtures thereof.

Non-limiting examples of preservatives include ethanol, polyvinyl alcohol, phenoxyethanol, benzyl alcohol, methyl paraben, propyl paraben and mixtures thereof.

Non-limiting examples of pH adjusting agents includes potassium acetate, sodium carbonate, sodium hydroxide, phosphoric acid, succinic acid, sodium citrate, citric acid, boric acid, lactic acid, sodium hydrogen carbonate and mixtures thereof.

Bleaching agents include, but not limited to, hydrogen peroxide, perborate and persufate salts. EDTA and other aminocarboxylates may be used as sequestering agents. Anti-dandruff agents such as zinc pyrithione, salicylic acid, climbazole, ketoconazole, sulfur piroctone olamine, selenium sulfide and mixtures thereof may also be used as an auxiliary ingredients.

The following examples are for illustrative purposes only and are not intended to limit the scope of the claims.

### EXAMPLE 1

### Amidation of Polyethylenimine (MW 1800 g/mol) with Meadowlactone

In a 100 ml stirred apparatus equipped with a distillation head, 10 g of polyethyleneimine (MW 1800 g/mol, Polysciences, Inc.) were heated to 50° C. Under nitrogen preheated 2.54 g of meadowlactone were added a little at a time. The mixture was then stirred at 65° C. for 4 hours. This provided a viscous water-soluble product. FT-IR (ThermoNicolet Nexus 470) absorbance spectra show the disappearance of the characteristic symmetric (C═O) band of the lactone at 1709 cm⁻¹ upon addition to polyethyleneimine, accompanied by the appearance of a typical secondary amide peak (C═O, amide I band) at 1650 cm⁻¹.

### EXAMPLE 2: Hair Styling Gel

| | Percentage |
|---|---|
| Ingredients | (%) |
| Deionized water | 93.80 |
| Sodium polystyrene sulfonate | 0.25 |
| Hydroxyethylcellulose | 0.55 |
| Hydroxypropyl guar | 0.30 |
| Meadowlactone amidated PEI_{1.8k} | 0.35 |
| Monopropylene glycol | 1.50 |
| Isostearic acid | 0.75 |
| Glycerine | 1.00 |
| Glyceryl stearate SE | 0.50 |
| Cetearyl alcohol | 0.50 |
| Dicaprylyl ether | 0.50 |

Meadowlactone amidated PEI_{1.8k}: 5% amidation of primary amines in polyethylencimine (1800 g/mol). Formula imparts on fine to average including sensitized hair with conditioning and styling effects, fast drying time, increased volume, body and control, excellent shine and water resistance.

### EXAMPLE 3: Shine Cream

| | Percentage |
|---|---|
| Ingredients | (%) |
| Deionized water | 89.20 |
| Medowlactone amidated PEI_{1.8k} | 0.30 |
| Petrolatum HIP emulsion | 3.00 |
| Isostearic acid | 0.50 |
| Hydogenated polyisobutene | 5.00 |
| Polyacrylamide | 2.00 |
| C₁₃₋₁₄ Isoparaffin | |
| Laureth-7 | |

Meadowlactone amidated PE_{1.8k}: 15% arnidation of primary amines in polyethyleneimine (1800 g/mol). Formula imparts on hair enhanced shine and water resistance.

### EXAMPLE 4: Tamer Cream

| | Percentage |
|---|---|
| Ingredients | (%) |
| Deionized water | 87.20 |
| DOW CORNING 2-1388 Emulsion | 5.00 |
| Polyquaternium-10 | 0.50 |
| Ditertiobutyl 4-hydroxytoluene | 0.10 |
| Meadowlactone amidated PEI_{1.8k} | 0.50 |
| Polyquaternium-37 | 4.00 |
| Mineral oil | |
| PPG-1 trideceth-6 | |
| Isostearic acid | 0.95 |
| Sodium methylparaben | 0.25 |
| Benzyl alcohol | 0.70 |
| Ethylparaben | 0.10 |
| Phenoxyethanol | 0.70 |

Meadowlactone amidated PEI_{1.8k}: 15% amidation of primary amines in polyethyleneimine (1800 g/mol). Formula tames unruly, frizzy hair and leaves hair smooth and conditioned from roots to ends. It further aids in detangling and imparts manageability and excellent shine.

### EXAMPLE 5: Curl Definer Cream

| | Percentage |
|---|---|
| Ingredients | (%) |
| Deionized water | 88.40 |
| Cyclopentasiloxane | 5.00 |
| Dimethiconol | |
| Laureth-23 | |
| Laureth-4 | |
| Polyquaternium-10 | 0.50 |
| Ditertiobutyl 4-hydroxytoluene | 0.10 |
| Meadowlactone amidated PEI_{1.8k} | 0.50 |
| Isostearic acid | 0.75 |
| Polyquaternium-37 | 3.00 |
| Mineral oil | |
| PPG-1 trideceth-6 | |
| Sodium methylparaben | 0.25 |
| Benzyl alcohol | 0.70 |
| Ethyl paraben | 0.10 |
| Phenoxyethanol | 0.70 |

Meadowlactone amidated PEI_{1.8k}: 15% amidation of primary amines in polyethyleneimine (1800 g/mol). Formula defines curls and tames frizz; rejuvenates permed hair and leaves hair smooth and shiny.

### EXAMPLE 6: Hair Styling Mousse

| | Percentage |
|---|---|
| Ingredients | (%) |
| Deionized water | 89.83 |
| Cetyl hydroxyethylcellulose | 0.38 |
| Sodium polystyrene sulfonate | 0.91 |
| Sodium benzoate | 0.48 |
| Meadowlactone amidated PEI_{1.8k} | 0.48 |
| Isostearic acid | 0.85 |
| Laureth-23 | 0.48 |
| Caprylyl glycol | 0.96 |
| Glycerin | 0.96 |
| Dimethicone PEG-7 phosphate | 0.19 |
| Cocamidopropyl betaine | 0.48 |
| Isobutane and propane (85/15) | 4.00 |

Meadowlactone amidated PEI_{1.8k}: 15% amidation of primary amines in polyethyleneimine (1800 g/mol). Formula provides hair with soft flexible hold without crispiness.

### EXAMPLE 7: Hair Styling and Shine Mousse

| | Percentage |
|---|---|
| Ingredients | (%) |
| Deionized water | 89.82 |
| Empicol BSD 52: | 0.38 |
| Sodium laureth-8 sulfate | |
| Magnesium laureth-8 sulfate | |
| Sodium laureth sulfate | |
| Magnesium laureth sulfate | |
| Sodium oleth sulfate | |
| Magnesium oleth sulfate | |
| Poly(dimethylaminopropyl methacrylamide) | 2.50 |
| Oleic acid | 0.65 |
| Mineral oil | 0.65 |
| Isobutane and propane (85/15) | 6.00 |

Formula provides hair with excellent cosmetic properties, e.g., flexible hold, texture, body and volume; smoothness, silky-feel and enhanced shine.

### EXAMPLE 8: Hair Shine Spray

| | Percentage |
|---|---|
| Ingredients | (%) |
| Denatured alcohol | 55.00 |
| Cyclopentasiloxane (DOW CORNING 245 Fluid) | 28.75 |
| Poly(2-ethyl-2-oxazoline)₈₅-co-Linear poly(ethyleneimine)₁₅ | 0.55 |
| Phenyl trimethicone | 10.00 |
| Isostearic acid | 0.70 |
| Octyldodecyl neopentanoate | 5.00 |

Formula provides hair with excellent long lasting shine, better feel and flexible hold.

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the disclosure in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

What is claimed:

1. A composition comprising
a. at least one polyamide,
b. at least one monoacid,
c. at least one water-insoluble ingredient and
d. solvent,
wherein the at least one polyamide is at least one selected from the group consisting of:
1) a polyamide formed by an amidation reaction between a polyamine and a lactone wherein the molar ratio of the amine groups of the polyamine to the carboxylate groups of the lactone is from about 2 to 1 to about 60 to 1,
2) a poly di-C₁₋₆-alkyl amino-C₁₋₆-alkyl (meth)acrylamide-co-poly-(poly-C₁₋₆-alkylglycol (meth)acrylate) polymer and
3) a poly (2-substituted-2-oxazoline) polymer wherein from about 3 to about 97 mol % of the amide groups in the poly (2-substituted-2-oxazoline) polymer are hydrolyzed to give amine groups.

2. The composition as claimed in claim 1, further comprising at least one auxiliary ingredient (e) wherein the at least one auxiliary ingredient is selected from the group consisting of amino acids, proteins, cationic conditioners, cationic polymers, anionic surfactants, nonionic surfactants, amphoteric surfactants, zwitterionic surfactants, viscosity modifiers, antibacterial agents, sunscreens, preservatives, pH adjusting agents, bleaching agents, perfumes, sequestering agents, anti-dandruff agents and mixtures thereof.

3. The composition as claimed in claim 1, wherein the polyamide 1) is utilized and wherein the polyamine is selected from the group consisting of a polyethyleneimine, a polyvinylamine, an aminated polysaccharide, an amine substituted polyalkylene glycol, an amine substituted polyacrylate crosspolymer, an amine substituted polyacrylate, an amine substituted polymethacrylate, a protein, an amine substituted polyester, a polyamino acid, an amodimethicone, a polyalkylamine, diethylene triamine, triethylenetetramine, spermidine, spermidine, aminosilicone and mixtures thereof.

4. The composition as claimed in claim 3, wherein the polyamine is selected from the group consisting of a polyethyleneimine, a polyvinylamine, chitosan, polylysine, carbopol® aqua CC and mixtures thereof.

5. The composition as claimed in claim 1, wherein the polyamide 1) is utilized and wherein the lactone is at least one selected from the group consisting of butyrolactone, butyrolactonethiol, gamma-caprolactone, delta-decalactone, gamma-decalatone, ethyl butyl valerolactone, galactonolactone, glucarolactone, glucoheptonolactone, gluconolactone, glucuronolactone, hexadecanolactone, meadowfoam delta-lactone, mevalonolactone, gamma-nonalactone, pantolactone, pentadecalactone, ribonolactone and gamma-undecalactone.

6. The composition as claimed in claim 1, wherein polyamide 2) is utilized and the polyamide is poly(dimethylaminopropyl(meth)acrylamide)-co-poly(polyethylene glycol (meth)acrylate) polymer.

7. The composition as claimed in claim 1, wherein polyamide 3) is utilized and the poly(2-substituted-2-oxazoline) polymer is poly(2-ethyl-2-oxazoline) polymer or poly (2-phenyl-2-oxazoline) polymer or a mixture of both.

8. The composition as claimed in claim 1, wherein the at least one monoacid (b) is selected from the group consisting of a fatty carboxylic acid, a fatty ether carboxylic acid, a fatty ether phosphoric acid, a fatty phosphoric acid and mixtures thereof.

9. The composition as claimed in claim 1, wherein the at least one monoacid (b) is selected from the group consisting of capric acid, caprylic acid, isostearic acid, oleic acid, stearic acid, lauric acid, linoueic acid, laureth-5 carboxylic acid, laureth-11 carboxylic acid, cetyl phosphosphate, stearyl phosphate, oleth-3 phosphate, oleth-10 phosphate and mixtures thereof.

10. The composition as claimed in claim 1, wherein the at least one water-insoluble ingredient (c) is selected from the group consisting of an oil, a fatty ester, a hydrocarbon oil, a silicone, a wax, a fatty acid, a fatty alcohol and mixtures thereof.

11. The composition as claimed in claim 1, wherein the at least one water-insoluble ingredient (c) is selected from the group consisting of olive oil, avocado oil, coconut oil, mineral oil, isopropyl palmitate, capric triglyceride, caprylic triglyceride, isododecane, polyisobutene, dimethicone phenylmethicone, beeswax and mixtures thereof.

12. The composition as claimed in claim 1, wherein the at least one polyamide (a) is present in a positive amount up to about 30% by weight, based on the weight of the composition.

13. The composition as claimed in claim 1, wherein the at least one monoacid (b) is present in a positive amount up to about 50% by weight, based on the weight of the composition.

14. The composition as claimed in claim 1, wherein the at least one water-insoluble ingredient (c) is present in a positive amount up to about 50% by weight, based on the weight of the composition.

15. The composition as claimed in claim 1, wherein solvent (d) is present in an amount of from about 10% to about 90% by weight, based on the weight of the composition.

16. The composition as claimed in claim 2, wherein the at least one auxiliary ingredient (e) is present in a positive amount up to about 50%, based on the weight of the coinposition.

17. A method of treating a keratinous substrate comprising contacting the keratinous substrate with a composition comprising
a. at least one polyamide,
b. at least one monoacid,
c. at least one water-insoluble ingredient and
d. solvent,
wherein the at least one polyamide is at least one selected from the group consisting of:
1) a polyamide formed by an amidation reaction between a polyamine and a lactone wherein the molar ratio of the amine groups of the polyamine to the carboxylate groups of the lactone is from about 2 to 1 to about 60 to 1,
2) a poly di-C₁₋₆-alkyl amino-C₁₋₆-alkyl (meth)acrylamide-co-poly-(poly-C₁₋₆ alkylglycol methacrylate) polymer and
3) a poly (2-substituted-2-oxazoline) polymer wherein from about 3 to about 97 mol % of the amide groups in the poly (2-substituted-2-oxazoline) polymer are hydrolyzed to give amine groups.

18. The method as claimed in claim 17, wherein the composition further comprises at least one auxiliary ingredient (e) wherein the at least one auxiliary ingredient is selected from the group consisting of amino acids, proteins, cationic conditioners, cationic polymers, anionic surfactants, nonionic surfactants, amphoteric surfactants, zwitterionic surfactants, viscosity modifiers, antibacterial agents, sunscreens preservatives, PH adjusting agents, bleaching agents, perfumes, sequestering agents, anti-dandruff agents and mixtures thereof.

19. The method as claimed in claim 17, wherein the polyamide 1) is utilized and wherein the polyamine is selected from the group consisting of a polyethyleneimine, a polyvinylamine, an aminated polysaccharide, an amine substituted polyalkylene glycol, an amine substituted polyacrylate crosspolymer, substituted polyacrylate, an amine substituted polymethacrylate, a protein, an amine substituted polyester, a polyamino acid, an amodimethicone, a polyalkylamine, diethylene triamine, triethylenetetramine, spermidine, spermine and mixtures thereof.

20. The method as claimed in claim 17, wherein the at least one monoacid (b) is selected from the group consisting of a fatty carboxylic acid, a fatty ether carboxylic acid, a fatty ether phosphoric acid, a fatty phosphoric acid and mixtures thereof.

21. The method as claimed in claim 17, wherein the at least one water-insoluble ingredient (c) is selected from the group consisting of an oil, a fatty ester, a hydrocarbon oil, a silicone, a wax, a fatty acid, a fatty alcohol and mixtures thereof.

22. The method as claimed in claim 17, wherein the at least one polyamide (a) is present in an amount of from greater than 0% to about 30% by weight, based on the weight of the composition.

23. The method as claimed in claim 17, wherein the at least one monoacid (b) is present in an amount of from greater than 0% to about 50% by weight, based on the weight of the composition.

24. The method as claimed in claim 17, wherein the at least one water-insoluble ingredient (c) is present in an amount of from greater than 0% to about 50% by weight, based on the weight of the composition.

25. The method as claimed in claim 17, wherein solvent (d) is present in an amount of from about 10% to about 90% by weight, based on the weight of the composition.

26. The method as claimed in claim 17, wherein the at least one auxiliary ingredient (e) is present in an amount from 0% to about 50%, based on the weight of the composition.

27. The method as claimed is claim 17, wherein the keratinous substrate is at least one selected from the group consisting of hair, skin, lips, nails and eyelashes.

28. The composition as claimed in claim 1, wherein the solvent is selected from the group consisting of water, ethanol, isopropanol and mixtures thereof.

29. The method as claimed in claim 17, wherein the solvent is selected from the group consisting of water, ethanol, isopropanol and mixtures thereof.

30. The composition as claimed in claim 1, wherein polyamide 3) is utilized and wherein the poly(2-substituted-2-oxazoline) polymer is substituted at the 2-position with a C₁-C₃₀ alkyl that may be a straight chain, branched or cyclic alkyl, a C₆-C₃₀ alkylaryl , a C₆-C₃₀ arylalkyl, a C₆-C₃₀ alkylheteroaryl, a C₆-C₃₀ heteroarylalkyl, a phenyl group optionally substituted with one or more C₁-C₆ alkyl groups, a C₁-C₃₀ alkanol, a C₁-C₃₀ alkylcarboxylic acid and its alkali metal salts, a C₁-C₃₀ alkylpolyol, a C₁-C₃₀ alkylquaternary amine, a Cl-C₃₀ alkylsulfonate, a C₁-C₃₀ alkylsulfate, a C₁-C₃₀ alkylphosphonate, a C₁-C₃₀ alkyl phosphate, a polysiloxane group or an amino acid group.

31. The method as claimed in claim 17, wherein polyamide 3) is utilized and wherein the poly(2-substituted-2-oxazoline) polymer is substituted at the 2-position with a C₁-C₃₀ alkyl that may be a straight chain, branched or cyclic alkyl, a C₆-C₃₀ alkylaryl 1, a C₆-C₃₀ arylalkyl, a C₆-C₃₀ alkylheteroaryl, a C₆-C₃₀ heteroarylalkyl, a phenyl group optionally substituted with one or more C₁-C₆alkyl groups, a C₁-C₃₀ alkanol, a C₁-C₃₀ alkylcarboxylic acid and its alkali metal salts, a C₁-C₃₀ alkylpolyol, a C₁-C₃₀ alkylquaternary amine, a C₁-C₃₀ alkylsulfonate, a C₁-C₃₀ alkylsulfate, a C₁-C₃₀ alkylphosphonate, a C₁-C₃₀ alkyl phosphate, a polysiloxane group or an amino acid group.
